(19) United States
(12) Patent Application Publication
(10) Pub. No.: US 2025/0228513 A1
(43) Pub. Date: Jul. 17, 2025

(54) NUCLEAR MEDICINE DIAGNOSIS APPARATUS AND METHOD

(71) Applicant: CANON MEDICAL SYSTEMS CORPORATION, Tochigi (JP)

(72) Inventors: Akshay Narayan Prabhu VERLEKER, Nasushiobara (JP); Shogo ONO, Utsunomiya (JP); Wenyuan QI, Vernon Hills, IL (US)

(73) Assignee: CANON MEDICAL SYSTEMS CORPORATION, Tochigi (JP)

(21) Appl. No.: 19/015,022

(22) Filed: Jan. 9, 2025

Related U.S. Application Data
(60) Provisional application No. 63/620,376, filed on Jan. 12, 2024.

(51) Int. Cl. A61B 6/00; A61B 6/03; G06T 11/00

(52) U.S. Cl. CPC A61B 6/5217; A61B 6/037; A61B 6/5288; G06T 11/005; G06T 2210/41

(57) ABSTRACT

A nuclear medicine diagnosis apparatus according to an embodiment includes processing circuitry configured: to generate a feature vector for each of the mini frames from list mode data acquired by performing a nuclear medicine scan and to calculate a Euclidean distance between each of the feature vectors corresponding to frames and a reference phase vector; to calculate, on the basis of the Euclidean distances, a mean Euclidean distance for a plurality of conditions obtained by varying a percentage of counts to be used in a reconstructing process and to determine the percentage on the basis of a relationship between the percentage and the mean Euclidean distance; and to generate a gated image by carrying out an image reconstruction while using the determined percentage as a gating condition.

### CROSS-REFERENCE TO RELATED APPLICATIONS

The present application relates to and claims priority under 35 U.S.C. § 119(e) to U.S. Provisional Patent Application No. 63/620,376 filed on Jan. 12, 2024, the contents of which are incorporated herein by reference.

### FIELD

Embodiments described herein relate generally to a nuclear medicine diagnosis apparatus and a method.

### BACKGROUND

Data-driven deviceless gating is a method by which a respiratory gated image is reconstructed without the need to use an external motion sensor, so that motion blur can be reduced. Conventionally, to carry out such data-driven deviceless gating, users need to select a gating condition.

### BRIEF DESCRIPTION OF THE DRAWINGS

FIG. 1 is a block diagram illustrating an exemplary configuration of a nuclear medicine diagnosis apparatus according to an embodiment;

FIG. 2 is a drawing illustrating an outline of processes performed by the nuclear medicine diagnosis apparatus according to the embodiment;

FIG. 3A is a chart illustrating conditions related to an image quality evaluation of clinical data using a count optimization method according to the embodiment;

FIG. 3B is another chart illustrating the conditions related to the image quality evaluation of the clinical data using the count optimization method according to the embodiment;

FIG. 3C is yet another chart illustrating the conditions related to the image quality evaluation of the clinical data using the count optimization method according to the embodiment;

FIG. 3D is yet another chart illustrating the conditions related to the image quality evaluation of the clinical data using the count optimization method according to the embodiment;

FIG. 3E is yet another chart illustrating the conditions related to the image quality evaluation of the clinical data using the count optimization method according to the embodiment;

FIG. 3F is a drawing illustrating the conditions related to the image quality evaluation of the clinical data using the count optimization method according to the embodiment;

FIG. 3G is a table illustrating the conditions related to the image quality evaluation of the clinical data using the count optimization method according to the embodiment;

FIG. 4A is a chart illustrating a result of the image quality evaluation of the clinical data using the count optimization method according to the embodiment;

FIG. 4B is a chart illustrating another result of the image quality evaluation of the clinical data using the count optimization method according to the embodiment;

FIG. 4C is a chart illustrating yet another result of the image quality evaluation of the clinical data using the count optimization method according to the embodiment;

FIG. 4D is a chart illustrating yet another result of the image quality evaluation of the clinical data using the count optimization method according to the embodiment;

FIG. 4E is a chart illustrating yet another result of the image quality evaluation of the clinical data using the count optimization method according to the embodiment;

FIG. 4F is a table illustrating results of the image quality evaluation of the clinical data using the count optimization method according to the embodiment;

FIG. 5 is a drawing illustrating results of the image quality evaluation of the clinical data using the count optimization method according to the embodiment;

FIG. 6 is a chart illustrating an example of a % Count determination method according to the embodiment; and

FIG. 7 is a drawing illustrating examples of the % Count determination method according to the embodiment.

### DETAILED DESCRIPTION

A nuclear medicine diagnosis apparatus according to an embodiment includes processing circuitry configured: to generate a feature vector (also called characteristic vectors) for each of the mini frames from list mode data acquired by performing a nuclear medicine scan and to calculate a Euclidean distance between each of the feature vectors corresponding to frames and a reference phase vector; to calculate, on the basis of the Euclidean distances, a mean Euclidean distance for a plurality of conditions obtained by varying a percentage of counts to be used in a reconstructing process and to determine the percentage on the basis of a relationship between the percentage and the mean Euclidean distance; and to generate a gated image by carrying out an image reconstruction while using the determined percentage as a gating condition.

Exemplary embodiments of a nuclear medicine diagnosis apparatus and a method will be explained below, with reference to the accompanying drawings.

### Embodiments

A nuclear medicine diagnosis apparatus 10 according to an embodiment is a medical image diagnosis apparatus (a modality) capable of performing a nuclear medicine scan. The nuclear medicine scan is a scan performed by administering a drug labeled with a radionuclide for an examined subject (hereinafter, “patient”). Typical examples thereof include a Positron Emission Computed Tomography (PET) scan and a Single Photon Emission Computed Tomography (SPECT) scan.

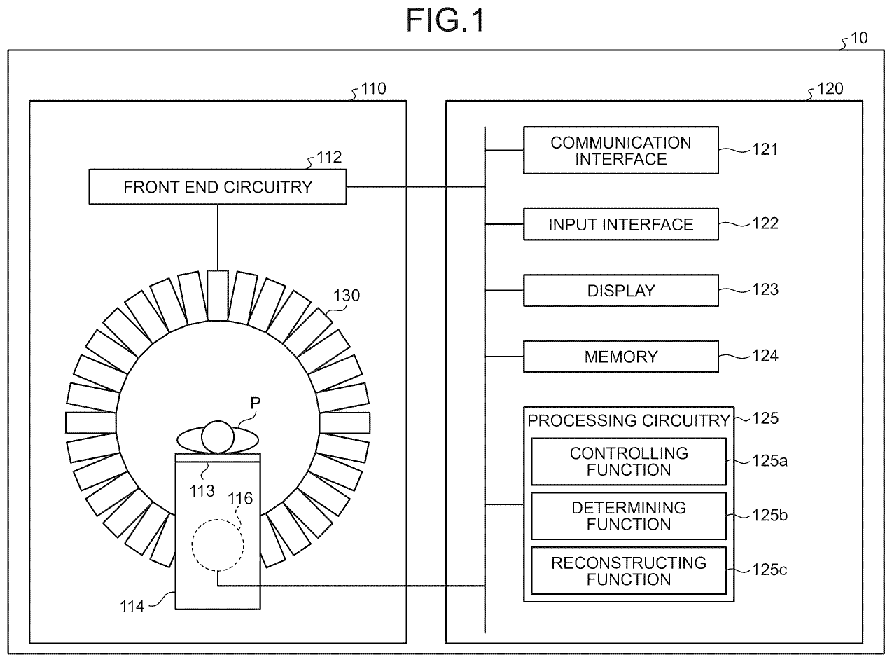

In the present embodiment, an example in which the nuclear medicine diagnosis apparatus 10 is a PET apparatus will be explained, with reference to FIG. 1. FIG. 1 is a diagram illustrating an exemplary configuration of the nuclear medicine diagnosis apparatus 10 according to the embodiment. The nuclear medicine diagnosis apparatus 10 illustrated in FIG. 1 includes a gantry apparatus 110 and a console apparatus 120. The gantry apparatus 110 includes detectors 130, front end circuitry 112, a tabletop 113, a table 114, and a table driving unit 116.

The detectors 130 are detectors configured to detect radiation. For example, the detectors 130 are detectors configured to detect the radiation, by detecting scintillation light (fluorescent light) which is the light re-released when a substance transitions back into a ground state after being in an excited state due to an interaction between a light emitting body and gamma rays generated from pair annihilation between positrons released from the drug administered for and accumulated in the patient P and electrons in a surrounding tissue. Further, in an embodiment, the detectors 130 are also capable of detecting Cherenkov light. The detectors 130 are configured to detect energy information of the radiation of the gamma rays generated from the pair annihilation between the positrons released from the drug administered for and accumulated in the patient P and the electrons in the surrounding tissue. The plurality of detectors 130 are arranged so as to surround the patient P in a ring formation and form a plurality of detector blocks, for example.

The detectors 130 typically include scintillator crystals and a light detecting surface structured with light detecting elements. As for a material of the scintillator crystals, it is possible to use, for example, a material suitable for generating Cherenkov light such as Bismuth Germanium Oxide (BGO) or a lead compound such as lead glass (SiO2+PbO), lead fluoride (PbF2), or PWO (PbWO4), for instance. Further, in other examples, it is also acceptable to use, for instance, scintillator crystals of Lutetium Yttrium Oxyorthosilicate (LYSO), Lutetium Oxyorthosilicate (LSO), Lutetium Gadolinium Oxyorthosilicate (LGSO), or BGO, for example. The light detecting elements structuring the light detecting surface represent a plurality of pixels, for example. Each of the pixels is structured with a Single Photon Avalanche Diode (SPAD), for example. Further, possible configurations of the detectors 130 are not limited to the above examples. In another example, the light detecting elements may be configured by using Silicon Photomultipliers (SiPMs) or photomultiplier tubes, for instance. Also, the scintillator crystals may be a monolithic crystal. Furthermore, light detecting surfaces structured with the light detecting elements may be arranged on the six faces of the scintillator crystals, for example.

Further, the gantry apparatus 110 is configured, by employing the front end circuitry 112, to generate count (count value) information from output signals of the detectors 130 and to store the generated count information into a memory 124 of the console apparatus 120. In an example, the detectors 130 may be divided into the plurality of blocks and provided with the front end circuitry 112.

The front end circuitry 112 is configured to generate the count information by converting the output signals of the detectors 130 into digital data. The count information includes, for example, detection positions, energy values, and detection times of annihilation gamma rays. For example, the front end circuitry 112 is configured to identify a plurality of light detecting elements that converted scintillation light into electrical signals with mutually the same timing. Further, the front end circuitry 112 is configured to identify scintillator numbers (P) indicating the positions of the scintillators to which the annihilation gamma rays became incident. A means for identifying the scintillator positions at which the annihilation gamma rays became incident may be configured to identify the positions by performing a center-of-gravity calculation on the basis of the positions of the light detecting elements and intensities of the electrical signals. Further, when the element sizes of the scintillators correspond to the element sizes of the light detecting elements, for example, the scintillator corresponding to a light detecting element that exhibited the largest output may be presumed as a scintillator position at which the annihilation gamma rays became incident, so as to eventually identify the scintillator position, for example, by further taking scattering between the scintillators into consideration.

Further, the front end circuitry 112 is configured to identify energy values (E) of the annihilation gamma rays that became incident to the detectors 130, by either performing an integral calculation on the intensities of the electrical signals output from the light detecting elements or measuring time (Time Over Threshold) required of the electrical signal intensities to exceed a threshold value. Further, the front end circuitry 112 is configured to identify detection times (T) at which the scintillation light from the annihilation gamma rays was detected by the detectors 130. In this situation, the detection times (T) may be absolute times or may be elapsed time periods since the start of an imaging process. As explained herein, the front end circuitry 112 is configured to generate the count information including the scintillator numbers (P), the energy values (E), and the detection times (T).

In this situation, for example, the front end circuitry 112 may be realized by using a Central Processing Unit (CPU), a Graphical Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)).

The tabletop 113 is a bed on which the patient P is placed and is arranged over the table 114. The table driving unit 116 is configured to move the tabletop 113 under control of a controlling function 125a of processing circuitry 125. For example, the table driving unit 116 is configured to move the patient P to the inside of an imaging opening of the gantry apparatus 110, by moving the tabletop 113.

The console apparatus 120 is configured to control the execution of the nuclear medicine scan by receiving operations performed on the nuclear medicine diagnosis apparatus 10 by an operator and to also reconstruct a nuclear medicine image from acquired list mode data. When the nuclear medicine diagnosis apparatus 10 is a PET apparatus, the console apparatus 120 is configured to control execution of a PET scan and to reconstruct a PET image from the list mode data. As illustrated in FIG. 1, the console apparatus 120 includes a communication interface 121, an input interface 122, a display 123, the memory 124, and the processing circuitry 125.

The communication interface 121 is configured to control transfer of various types of data and communication transmitted and received between the console apparatus 120 and other apparatuses and systems connected via a network. More specifically, the communication interface 121 is connected to the processing circuitry 125 and is configured to output any of the data received from the other apparatuses and systems to the processing circuitry 125 and to also transmit data output from the processing circuitry 125 to any of the other apparatuses and systems. For example, the communication interface 121 is realized by using a network card, a network adaptor, a Network Interface Controller (NIC), or the like.

The input interface 122 is configured to receive various types of input operations from the operator, to convert the received input operations into electrical signals, and to output the electrical signals to the processing circuitry 125. For example, the input interface 122 is realized by using a mouse, a keyboard, a trackball, a switch, a button, a joystick, a touchpad on which input operations can be performed by touching an operation surface thereof, a touch screen in which a display screen and a touchpad are integrally formed, contactless input circuitry using an optical sensor, audio input circuitry, and/or the like. Alternatively, the input interface 122 may be configured by using a tablet terminal or the like capable of wirelessly communicating with a main body of the console apparatus 120. Further, the input interface 122 may be circuitry configured to receive input operations from the operator via a motion capture scheme. In an example, the input interface 122 is capable of receiving, as the input operations, body movements, lines of sight, and the like of the operator, by processing signals obtained via a tracker or images acquired of the operator. Further, the input interface 122 does not necessarily need to include physical operation component parts such as the mouse, the keyboard, and/or the like. For instance, possible examples of the input interface 122 include electrical signal processing circuitry configured to receive an electrical signal corresponding to an input operation from an external input mechanism provided separately from the console apparatus 120 and to output the electrical signal to the processing circuitry 125.

The display 123 is configured to display various types of information. For example, the display 123 is configured to display various types of medical information such as the nuclear medicine image acquired of the patient P, a respiratory waveform, and a heartbeat waveform. Further, for example, the display 123 is configured to display a Graphical User Interface (GUI) used for receiving various types of instructions, settings, and the like from the operator via the input interface 122. For example, the display 123 may be a liquid crystal display or a Cathode Ray Tube (CRT) display. The display 123 may be of desktop type or may be configured by using a tablet terminal or the like capable of wirelessly communicating with the main body of the console apparatus 120.

The memory 124 is realized by using, for example, a semiconductor memory element such as a Random Access Memory (RAM) or a flash memory, a hard disk, an optical disc, or the like. For example, the memory 124 is configured to store therein various types of medical information and a program used by the circuitry included in the console apparatus 120 to realize the functions thereof. In an example, the memory 124 may be realized by using a server group (a cloud) connected to the console apparatus 120 via a network NW.

The processing circuitry 125 is configured to control operations of the entirety of the console apparatus 120, by functioning as the controlling function 125a, a determining function 125b, and a reconstructing function 125c. For example, the processing circuitry 125 is configured to function as the controlling function 125a, by reading and executing a program corresponding to the controlling function 125a from the memory 124.

For example, the controlling function 125a is configured to carry out the nuclear medicine scan for the patient P by controlling operations of various types of configurations included in the gantry apparatus 110 and to thereby obtain the list mode data indicating a result of the radiation detection. Further, the controlling function 125a is configured to exercise display control on the display 123. In addition, the controlling function 125a is configured to control the transmission and reception of various types of data performed via a network. For example, the controlling function 125a is configured to transmit and register, into a Picture Archiving and Communication System (PACS) server, the list mode data acquired through the nuclear medicine scan and the nuclear medicine image reconstructed on the basis of the list mode data.

Similarly, the processing circuitry 125 is configured to function as the determining function 125b and the reconstructing function 125c. The controlling function 125a is an example of a controlling unit. The determining function 125b is an example of a determining unit. The reconstructing function 125c is an example of a reconstructing unit.

In the nuclear medicine diagnosis apparatus 10 illustrated in FIG. 1, the processing functions are stored in the memory 124 in the form of computer-executable programs. The processing circuitry 125 is a processor configured to realize the functions corresponding to the programs by reading and executing the programs from the memory 124. In other words, the processing circuitry 125 that has read the programs has the functions corresponding to the read programs.

Although the example was explained with reference to FIG. 1 in which the single piece of processing circuitry (the processing circuitry 125) is configured to realize the controlling function 125a, the determining function 125b, and the reconstructing function 125c, it is also acceptable to structure the processing circuitry 125 by combining together a plurality of independent processors, so that the functions are realized as a result of the processors executing the programs. Further, the processing functions included in the processing circuitry 125 may be realized as being distributed among or integrated together into one or more pieces of processing circuitry as appropriate.

Alternatively, the processing circuitry 125 may be configured to realize the functions, by using a processor of an external apparatus connected via the network NW. In an example, the processing circuitry 125 may be configured to realize the functions illustrated in FIG. 1, by reading and executing the programs corresponding to the functions from the memory 124, while using a server group (a cloud) connected to the nuclear medicine diagnosis apparatus 10 via the network NW as a computation resource.

An exemplary configuration of the nuclear medicine diagnosis apparatus 10 has thus been explained. In this situation, the list mode data acquired through the nuclear medicine scan may be impacted by respiratory movements. Further, as a method for reducing motion blur that may be caused by such respiratory movements, respiratory gating is known. Methods for realizing the respiratory gating include a device-based method using an external mechanism and a deviceless method using no external mechanism.

For the device-based respiratory gating, at the time of executing a nuclear medicine scan, the external mechanism such as a respiration synchronized monitor is attached to the patient. With this arrangement, it is possible to measure a respiratory waveform of the patient with the external mechanism while acquiring the list mode data and to bring the list mode data into correspondence with the respiratory waveform. It should be noted, however, that the device-based respiratory gating has various disadvantages such as degradation of workflows, cost increases, and impacts on the patient's skin.

The deviceless respiratory gating has conventionally been carried out with various types of gating conditions that are selected by users. With previous studies, the present inventors proposed a method for automatically selecting a quiescent respiratory phase on the basis of a feature motion vector calculation. According to this proposal, a user's selection is received regarding a count.

More specifically, at the time of carrying out the respiratory gating, the more count values among all the acquired data are used for a reconstructing process, the less noise there will be, and the better an SD value will be. However, the more count values are used, the more easily movements such as respiratory movements will exert an impact, and the blurrier the image will become. As a result of the image becoming blurrier, the contrast of a site (i.e., a tumor) which the user wishes to observe may be degraded in some situations.

For this reason, it is necessary to appropriately set, as a gating condition, how many count values are to be used in the reconstructing process, while taking into consideration balance between the noise and the blur. In the following sections, the gating condition indicating a percentage of the counts to be used in the reconstructing process may be referred to as % Counts. As mentioned above, the % Counts have conventionally been based on the user's selection.

As for methods for automating the control exercised on the % Counts, possible methods include using a recommended fixed value; however, appropriate % Counts may vary depending on the position of the bed, the patient's respiratory motion (fast, slow, irregular, etc.), the radiation dose, Body Mass Index (BMI) of the patient, and the like. Consequently, it would be difficult to appropriately cover multiple patients with a single recommended value for the % Counts. The most important factor which affects the optimal count selection is the patient's respiratory motion (fast breathing/slow breathing/asymmetric breathing cycles, etc.) which varies between patient to patient. The optimal % counts are small when the quiescent phase (low motion) is short (e.g., in fast breathing/irregular breathing) and optimal % count is higher if the quiescent phase is long (e.g., in slow and regular breathing).

The nuclear medicine diagnosis apparatus 10 according to an embodiment is configured to appropriately carry out automatic count control in the deviceless respiratory gating, so as to improve a clinical workflow while ensuring image quality by reducing the blur that may be caused by respiratory movements. A count optimization algorithm according to the embodiment optimizes counts generally by detecting a steep transition in motion characteristics from an expiration phase to an inspiration phase.

This algorithm makes use of the motion characteristics of the patient that are represented by feature vectors derived from short duration (mini frame) PET images reconstructed in correspondence with different phases in the respiratory cycle, as also described in our previous study. The supra-second duration of the mini-frame image is small enough to capture variation in respiratory motion, but large enough to exclude cardiac motion of sub-second duration (through averaging using overlapping durations).

At first, a reference phase vector is calculated by averaging the vectors corresponding to quiescent phases across all the respiratory cycles. In this regard, the reference phase vector may simply be referred to as a reference vector. Next, with respect to vectors for the respiratory phase, an Euclidean distance from the reference vector is calculated.

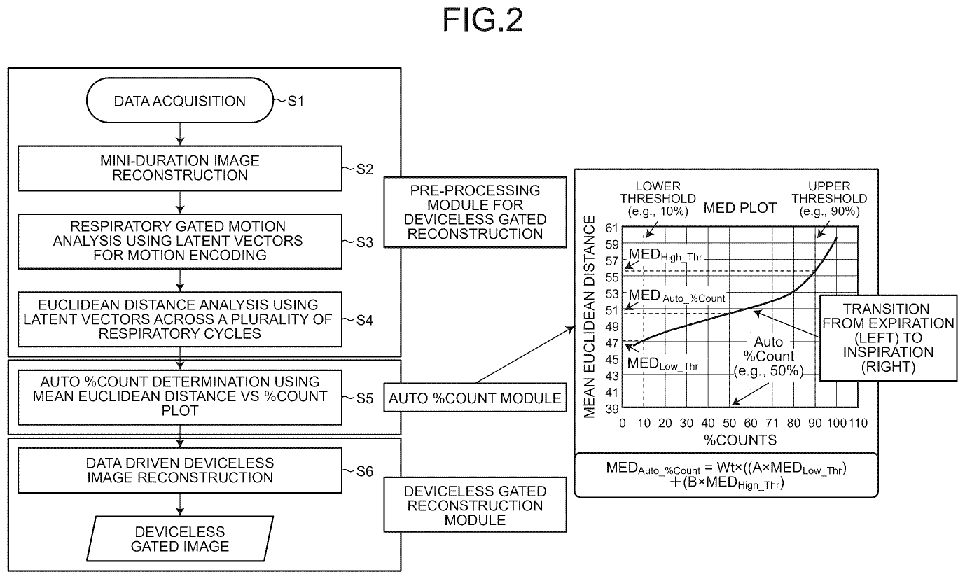

Subsequently, the Euclidean distances are then sorted from low to high, and a Mean Euclidean Distance (MED) is calculated for each % Count (e.g., from “5%” to “100%”). An optimal % Count is selected from a plot of “MED vs % Counts”, by detecting the steep transition in the MED (motion change) from the expiration phase to the inspiration phase. To be more general, the optimal % Count can be selected by modelling and analyzing the MED as a function of counts. As presented in the right section of FIG. 2, it is possible to express the plot as an MED curve. In a multiple-bed acquisition, an optimal % Count among all the beds is selected from the individual MED curves corresponding to the different beds and then applied across all the beds.

### A

Alternatively, additional criteria can be applied to select the optimal count for each bed such as: (a) detecting maximum respiratory motion (MED curve with the steepest transition) among the MED curves for all beds; (b) user can specify several beds and the criteria based on which the algorithm can select the optimal counts for each bed; or (c) using different optimal % counts for each bed by using automated anatomical identification techniques (e.g., AI based segmentation) to identify optimal counts for each bed (e.g., identify lung region, liver region) and select optimal counts for each region based on anatomical motion of one or several organs.

FIG. 2 illustrates a specific example of the processes described above. The count optimization method presented in FIG. 2 reduces motion blur by detecting the steep transition in the MED from the expiration phase to the inspiration phase, as described above. The % Count was optimized by modeling the MED as a mathematical function of weights (Wt, A, and B) derived from the MED curve (with upper and lower count threshold).

The “preprocessing module for deviceless gated reconstruction” presented in FIG. 2 corresponds to the controlling function 125a and carries out steps S1 through S4. Further, the “auto % Count module” presented in FIG. 2 corresponds to the determining function 125b and carries out step S5. In addition, the “deviceless gated reconstruction module” presented in FIG. 2 corresponds to the reconstructing function 125c and carries out step S6.

At first, the processing circuitry 125 carries out the nuclear medicine scan and acquires the list mode data (step S1).

Subsequently, the processing circuitry 125 performs a short duration image reconstruction with respect to the list mode data (step S2). For example, the processing circuitry 125 sets a duration of a certain length that makes it possible to capture variation in the respiratory motion while being able to exclude the cardiac motion and further reconstructs a mini-frame image with respect to each of the mini frames corresponding to the duration.

After that, the processing circuitry 125 encodes motion from a respiratory gated motion analysis, by using a latent vector (step S3).

In other words, the processing circuitry 125 quantifies the respiratory motion in each of the respiratory phases, by generating the latent vectors from the mini-frame images reconstructed at step S2. Although the type of the encoder is not particularly limited, it is possible to use, in an example, an autoencoder based on a neural network that carries out a Principal Component Analysis (PCA) and a waveform analysis. The latent vectors are examples of the feature vectors.

Subsequently, the processing circuitry 125 performs a Euclidean distance analysis using the latent vectors over the plurality of respiratory cycles (step S4).

For example, the processing circuitry 125 calculates the reference phase vector by extracting the latent vector corresponding to the quiescent phase in each of the plurality of respiratory cycles and averaging the extracted latent vectors. Further, the processing circuitry 125 calculates a Euclidean distance between the latent vector in each of the frames (i.e., at each of different points in the respiratory waveform) and the reference phase vector.

The Euclidean distance is calculated on the basis of Expression (1) presented below, for example. In Expression (1), “lvₙ” denotes an n-dimensional point of each of the latent vectors (lv); “lv_refₙ” denotes an n-dimensional point of the reference phase vector (lv_ref); and “k” denotes the dimension number of the latent vector.

$$Euclidean Distance = \sqrt{{\sum }_{n = 1}^{k} {( {lv}_{n} - {lv\_ref}_{n} )}^{2}} \qquad ( 1 )$$

After that, the processing circuitry 125 automatically determines the % Count by using a plot of “MED vs % Counts” (step S5).

For example, to begin with, the processing circuitry 125 rearranges the Euclidean distances calculated at step S4 in the ascending order. In other words, the processing circuitry 125 aligns the pieces of data on the basis of similarity in the respiratory motion.

Further, the processing circuitry 125 calculates an MED with respect to each of the % Counts. In other words, on the basis of the Euclidean distances calculated at step S4, the processing circuitry 125 calculates the MED for a plurality of conditions obtained by varying the percentage of the counts to be used in the reconstructing process. As a result, the plot of “MED vs % Counts” is obtained. To be more general, the MED as a function of the counts is obtained. The plot of “MED vs % Counts”, or the MED as a function of the counts is an example of the relationship between the percentage of the counts to be used in the reconstructing process and the MED.

In this situation, the processing circuitry 125 is capable of determining a % center so as to minimize the MED with respect to each of the % Counts. The % center is a phase center corresponding to the phase (the quiescent phase) having the least motion and serves as a gating condition used in the reconstructing process at step S6.

Subsequently, the processing circuitry 125 generates an MED curve by performing a fitting process on the plot of “MED vs % Counts”. As a result, impacts of local non-uniformity are reduced. In addition, as also presented in FIG. 2, the processing circuitry 125 determines “MED_{Auto_% Count}” on the basis of Expression (2) presented below.

$${MED}_{Auto \_ \% Count} = Wt \times ( ( A \times {MED}_{Low \_ Thr} ) + ( B \times {MED}_{High \_ Thr} ) ) \qquad ( 2 )$$

More specifically, at first, the processing circuitry 125 sets the upper threshold “High_Thr” and the lower threshold “Low_Thr”. Subsequently, the processing circuitry 125 identifies the value of the MED corresponding to the upper threshold “High_Thr” on the MED curve as “MED_{High_Thr}”. Also, the processing circuitry 125 identifies the value of the MED corresponding to the lower threshold “Low_Thr” on the MED curve as “MED_{Low_Thr}”. After that, as indicated in Expression (2), the processing circuitry 125 is able to automatically determine a % Count by calculating a weighted average of “MED_{High_Thr}” and “MED_{Low_Thr}”.

In other words, from Expression (2), the MED point called “MED_{Auto_% Count}” is determined, and also, the % Count corresponding to “MED_{Auto_% Count}” is automatically determined. It is possible to optimize the selection of the MED point, by adjusting the weights “Wt”, “A”, and “B”.

The notation “Auto % Count” is a condition determined with respect to each bed. When there are a plurality of beds, the processing circuitry 125 is configured to determine “Auto % Count” with respect to each of the beds. For example, after determining “Auto % Count” with respect to arbitrary beds, the processing circuitry 125 is configured to judge whether or not there are one or more beds remaining for which “Auto % Count” has not been determined. If there is at least one remaining bed, “Auto % Count” is determined by repeatedly performing the processes described above. On the contrary, when “Auto % Count” has been determined with respect to all the beds, the processing circuitry 125 is configured to determine a % Count called “Final Auto % Count” to be applied to all the beds.

For example, the processing circuitry 125 may select a bed having the smallest “Auto % Count” as the “Final Auto % Count”. Although the magnitudes of the respiratory motion may vary among the beds, the “Final Auto % Count” is also capable of addressing a bed exhibiting the largest respiratory motion.

As explained above, the processing circuitry 125 is capable of automatically setting the various types of respiratory gating conditions including the % Count and the % center. In addition, the processing circuitry 125 generates a deviceless gated image, by performing a data-driven deviceless image reconstruction (step S6) on the basis of the respiratory gating condition that was set.

The series of processes presented in FIG. 2 are carried out in a deviceless manner, so that the workflow is improved as compared with the device-based respiratory gating. Furthermore, the processing circuitry 125 is capable of automatically carrying out the series of processes presented in FIG. 2, without the need to have the user perform the operation to select the gating condition. In this manner, the nuclear medicine diagnosis apparatus 10 according to the embodiment is able to improve the workflow related to the respiratory gating.

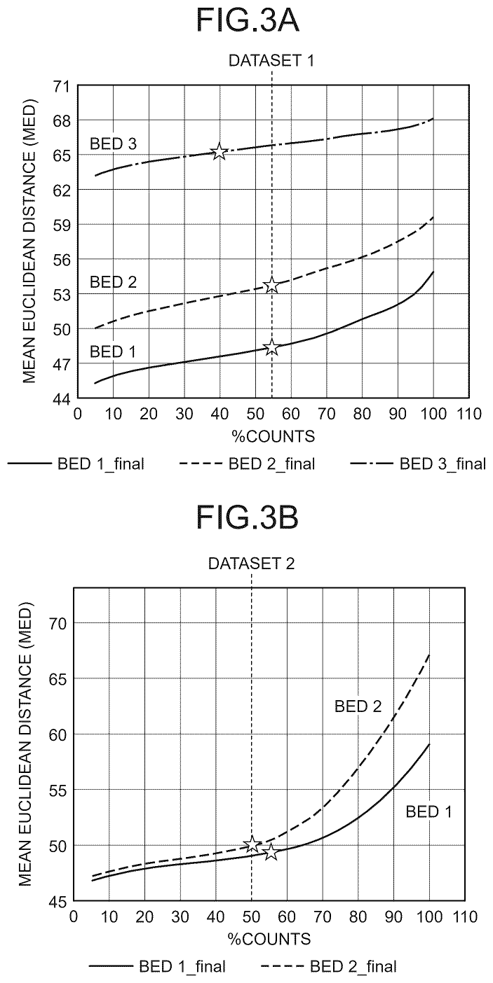

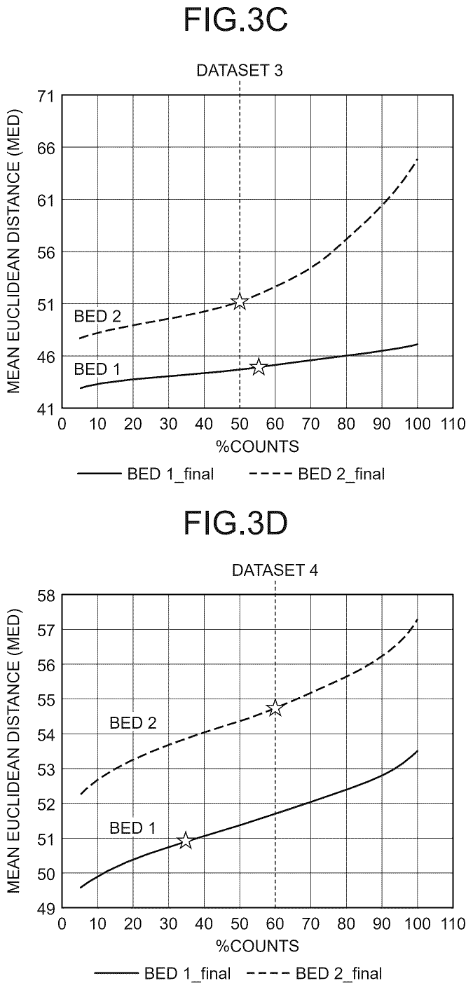

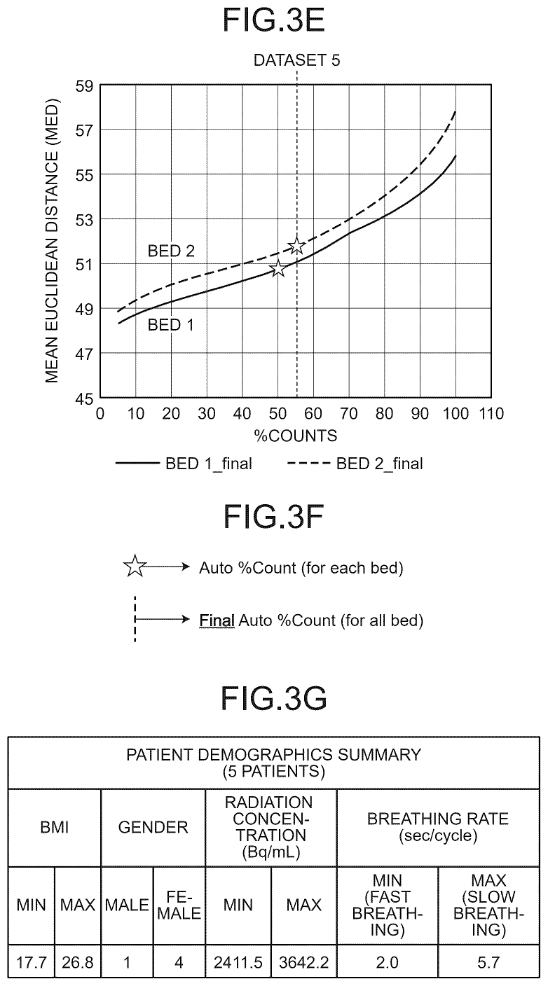

The present inventors evaluated image quality of five clinical datasets, by using the count optimization method described above. Conditions of the evaluation will be explained, with reference to FIGS. 3A to 3G. FIGS. 3A, 3B, 3C, 3D, and 3E present curves of “MED vs % Counts” with respect to the five clinical datasets acquired from a plurality of beds. Further, FIG. 3F presents a legend of the % Counts presented in the curves in FIGS. 3A to 3E. In other words, on the curves in FIGS. 3A to 3E, the star symbols each indicate a % Count (an Auto % Count) automatically determined with respect to each bed. For the curves in FIGS. 3A to 3E, the broken lines in FIG. 3 each present a % Count (the Final Auto % Count) to be applied to all the beds. FIG. 3G presents an outline of statistical information related to the patient subject to the evaluation.

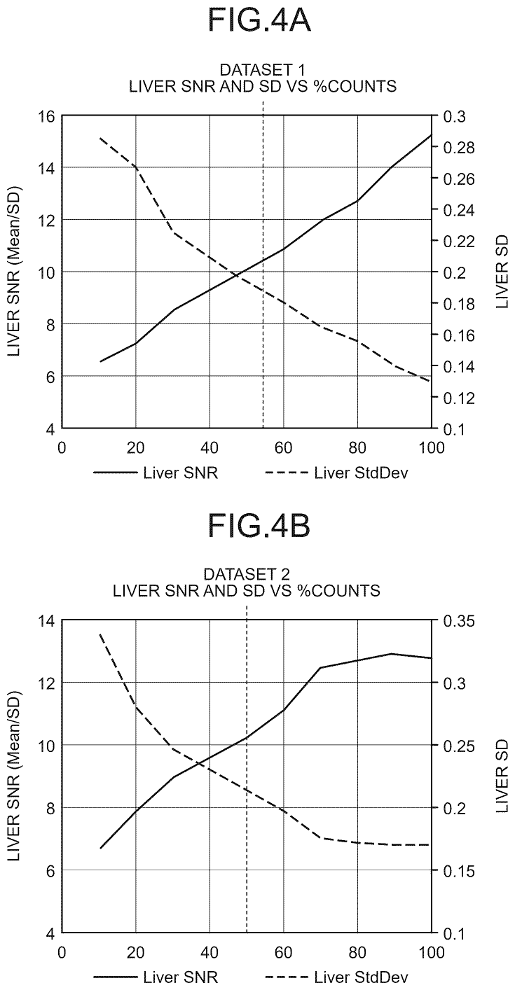

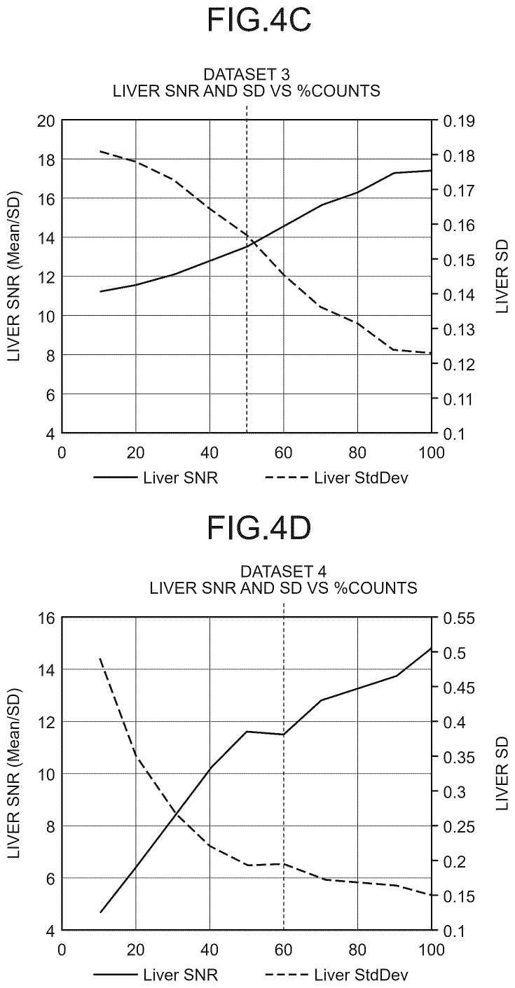

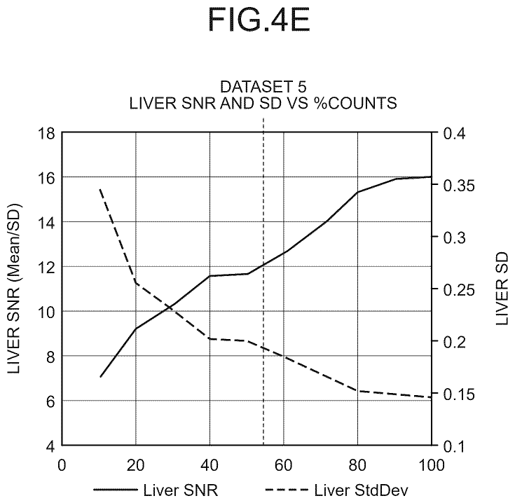

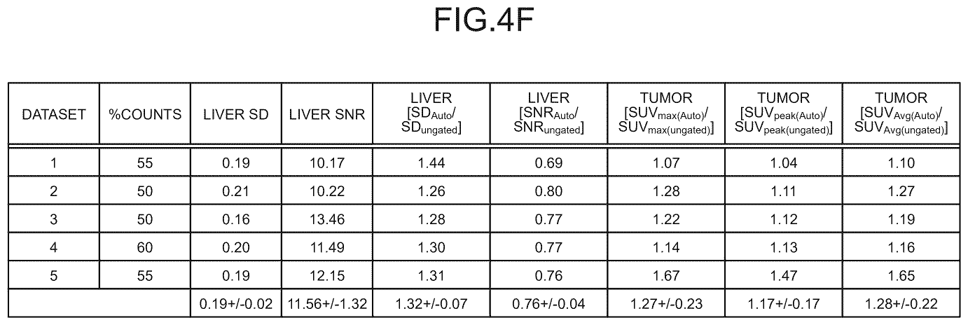

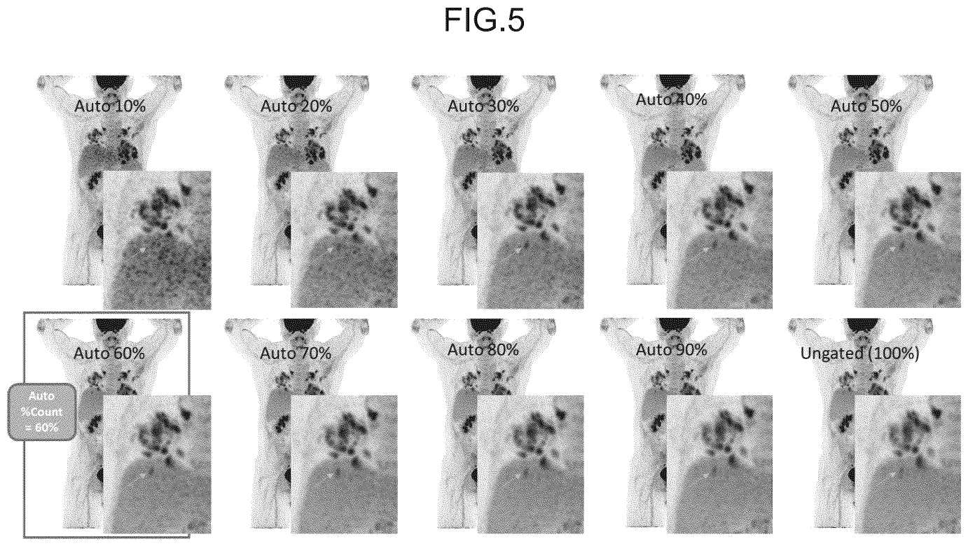

Further, FIGS. 4A to 4F and FIG. 5 present evaluation results. The plots in FIGS. 4A, 4B, 4C, 4D, and 4E each exhibit a decrease in the SD (Noise) and an increase in the SNR in the liver, with an increase in the counts. The count optimization achieved a stable ratio of SD and SNR in the liver, and tumor contrast (SUV) with respect to the ungated image (see the table in FIG. 4F).

As presented in FIG. 5, an increase in motion blur and a decrease in the liver SD are exhibited with an increase in the % Counts for Dataset 4. The method according to the embodiment selects a % Count (e.g., 60%) which achieves a balance between a liver IQ and the motion-related blurring.

The count optimization improved image quality with the reduced respiratory motion blur and with a stable ratio between the liver IQ and a tumor IQ as compared to the ungated image. More specifically, as presented in FIG. 4F, the liver IQ exhibited “SD_{Auto}/SD_{ungated}=0.19±0.02” and “SNR_{Auto}/SNR_{ungated}=11.56±1.32”. The tumor IQ exhibited “SUV_{max(Auto)}/SUV_{max(ungated)}=1.27±0.23”, “SUV_{peak(Auto)}/SUV_{peak(ungated)}=1.17±0.1”, and “SUV_{Avg(Auto)}/SUV_{Avg(ungated)}=1.28±0.22”, which are stable ratios.

The method for determining the % Count by using Expression (2) has thus been explained; however, possible embodiments are not limited to this example.

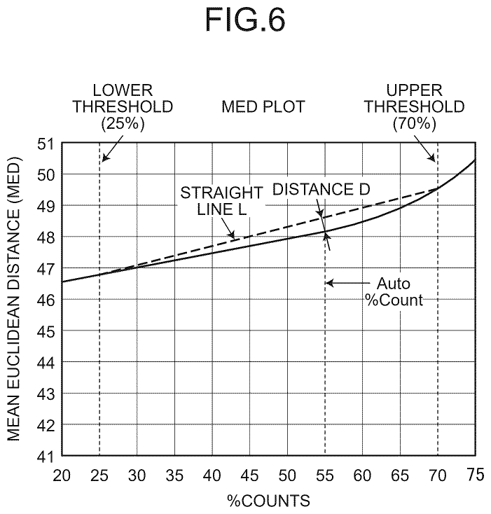

As presented in FIG. 6, the processing circuitry 125 may determine a % Count by identifying, on the MED curve, a point that maximizes the distance D from a straight line L to the MED curve.

More specifically, the processing circuitry 125 may generate the MED curve presented in FIG. 6, by performing a fitting process on the plot of “MED vs % Counts”. In addition, the processing circuitry 125 may be configured to set an upper threshold and a lower threshold with respect to the % Counts. In the example in FIG. 6, the lower threshold is set to “25%”, whereas the upper threshold is set to “70%”. Further, the processing circuitry 125 is configured to set the straight line L passing through the point corresponding to the upper threshold on the MED curve and the point corresponding to the lower threshold on the MED curve. In addition, the processing circuitry 125 is configured to calculate the distances between different points on the MED curve and the straight line L, so as to identify the point that maximizes the distance on the MED curve. After that, the processing circuitry 125 is configured to determine the % Count corresponding to the identified point as the gating condition.

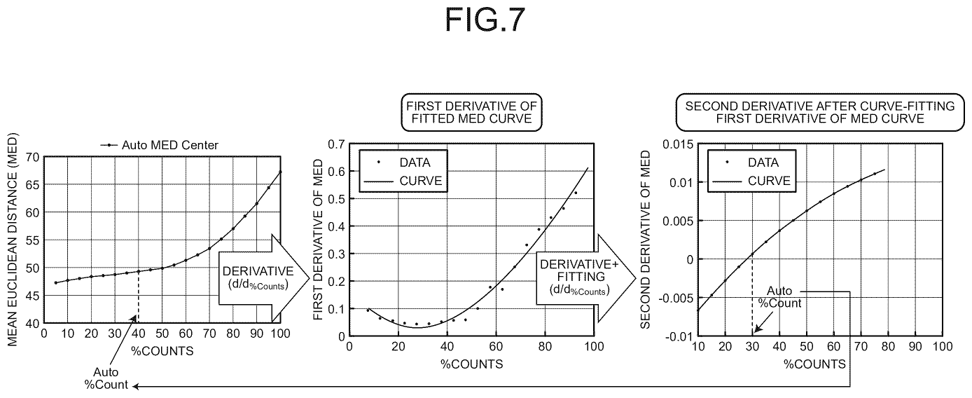

In another example, the processing circuitry 125 may be configured to determine a % Count by calculating a second derivative with respect to the MED curve, as presented in FIG. 7.

More specifically, the processing circuitry 125 may be configured to generate the MED curve presented in the left section of FIG. 7, by performing a fitting process on the plot of “MED vs % Counts”. Subsequently, the processing circuitry 125 may obtain the first derivative curve presented in the middle section of FIG. 7, by calculating a first derivative on the fitted MED curve and further apply curve fitting to the first derivative curve. Subsequently, the processing circuitry 125 may obtain the second derivative curve presented in the right section of FIG. 7, by calculating a second derivative on the curve-fitted first derivative curve. After that, the processing circuitry 125 is configured to determine a % Count on the basis of the position at which the second derivative curve is equal to “0”. For example, among the % Count values of which data is available, the processing circuitry 125 is configured to determine the value closest to the position at which the second derivative curve is equal to “0”, as the gating condition. The % Count determined in this manner indicates the first inflection point on the MED curve, i.e., the point at which the MED curve starts increasing exponentially.

As explained above, the processing circuitry 125 is capable of determining the % Counts by using the plurality of methods. In this regard, the processing circuitry 125 may select one of the plurality of methods described above or may use the plurality of methods described above in combination.

For example, the processing circuitry 125 may be configured to determine the % Count by using the method presented in FIG. 6. In the following sections, the % Count determined according to the method presented in FIG. 6 may be referred to “Count_bed_dist”. The notation “Count_bed_dist” is an example of the first percentage.

Alternatively, the processing circuitry 125 may be configured to determine the % Count by using the method presented in FIG. 7. In the following sections, the % Count determined according to the method presented in FIG. 7 may be referred to “Count_bed_der”. The notation “Count_bed_der” is an example of the second percentage.

After that, the processing circuitry 125 is configured to determine “MED_{Auto_% Count}”, by assigning “Count_bed_dist” and “Count_bed_der” to Expression (3) presented below. After that, as a result of the MED point called “MED_{Auto_% Count}” being determined, a % Count corresponding to “MED_{Auto_% Count}” will automatically be determined. In other words, the processing circuitry 125 is capable of automatically determining the % Count, by calculating a weighted average of the first percentage and the second percentage.

$${MED}_{Auto \_ \% Count} = Wt \times ( ( A \times Count\_bed \_dist ) + ( B \times Count\_bed \_der ) ) \qquad ( 3 )$$

It is possible to optimize the selection of the MED point by adjusting the weights “Wt”, “A”, and “B”. For example, when the user wishes to select an intermediate point between the method presented in FIG. 6 and the method presented in FIG. 7, weights such as “Wt=0.5”, “A=1”, and “B=1” are set.

Although the method was explained by which the % Counts and the % center are automatically set, the processing circuitry 125 is also capable of setting other conditions.

Examples of the other conditions include a post filter type and a strength. In an example, the processing circuitry 125 is capable of automatically setting a three-dimensional (3D) Gaussian filter with a Full-Width Half Maximum (FWHM) being “5 mm” and a Clear adaptive Low-noise Method (CaLM) being “strong”.

Another example of the other conditions is a reconstruction parameter to be used when a reconstructing process is performed by using Ordered Subsets Expectations Maximization (OSEM). Examples of the OSEM reconstruction parameters include an iteration number and the number of subsets.

Another example of the other conditions is a reconstruction parameter to be used when an AI-based reconstructing process is performed. Examples of the reconstruction parameter to be used for the AI-based reconstructing process include a network parameter and a filter strength.

The various types of conditions described above are adjusted to achieve optimal image quality based on the patient's BMI, a scan dose (i.e., an injected dose and scan delay time), a scan duration, and the like.

For example, the processing circuitry 125 may be configured to cumulatively quantify scan doses, acquisition time, BMI values, and the like. For example, the processing circuitry 125 is capable of using the Cumulative Dose Product (CDP), which is the product of concentration and duration, for quantifying the scan doses and acquisition durations. Further, the processing circuitry 125 is also capable of using a mathematical function of the CDP and the BMI (e.g., “f(CDP,BMI)=CDP/BMI”) as a metric. For example, for small “f(CDP,BMI)” values (i.e., more noise), the gating and reconstruction parameters may be optimized for a stronger noise suppression.

The term “processor” used in the above explanations denotes, for example, a CPU, a GPU, or circuitry such as an ASIC or a programmable logic device (e.g., a Simple Programmable Logic Device (SPLD), a Complex Programmable Logic Device (CPLD), or a Field Programmable Gate Array (FPGA)). When the processor is a CPU, for example, one or more processors are configured to realize the functions by reading and executing the programs saved in storage circuitry. In contrast, when the processor is an ASIC, for example, instead of having the programs saved in the storage circuitry, the functions are directly incorporated as logic circuitry in the circuitry of the one or more processors. Further, the processors in the present embodiments do not each necessarily have to be structured as a single piece of circuitry. It is also acceptable to structure one processor by combining together a plurality of pieces of independent circuitry so as to realize the functions thereof. Further, it is also acceptable to integrate two or more of the constituent elements illustrated in the drawings into one processor so as to realize the functions thereof.

The constituent elements of the apparatuses in the above embodiments are based on functional concepts. Thus, it is not necessarily required to physically configure the constituent elements as indicated in the drawings. In other words, specific modes of distribution and integration of the apparatuses are not limited to those illustrated in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a CPU and a program analyzed and executed by the CPU or may be realized as hardware using wired logic.

Further, it is possible to realize any of the methods explained in the above embodiments, by causing a computer such as a personal computer or a workstation to execute a program prepared in advance. The program may be distributed via a network such as the Internet. Further, the program may also be executed, as being recorded on a non-transitory computer-readable recording medium such as a hard disk, a flexible disk (FD), a Compact Disk Read-Only Memory (CD-ROM), a Magneto Optical (MO) disc, a Digital Versatile Disc (DVD), or the like and being read by a computer from the recording medium.

According to at least one aspect of the embodiments described above, it is possible to improve the workflow related to the respiratory gating.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A nuclear medicine diagnosis apparatus comprising processing circuitry configured:
to generate a feature vector for each of mini frames from list mode data acquired by performing a nuclear medicine scan and to calculate a Euclidean distance between each of feature vectors corresponding to frames and a reference phase vector;
to calculate, on a basis of the Euclidean distances, a mean Euclidean distance for a plurality of conditions obtained by varying a percentage of counts to be used in a reconstructing process and to determine the percentage on a basis of a relationship between the percentage and the mean Euclidean distance; and
to generate a gated image by carrying out an image reconstruction while using the determined percentage as a gating condition.

2. The nuclear medicine diagnosis apparatus according to claim 1, wherein the relationship is a Mean Euclidean Distance (MED) plot obtained by plotting values of the mean Euclidean distance with respect to the percentage.

3. The nuclear medicine diagnosis apparatus according to claim 2, wherein the processing circuitry is configured to determine the percentage, on a basis of a MED curve obtained by curve-fitting the MED plot.

4. The nuclear medicine diagnosis apparatus according to claim 3, wherein
the processing circuitry is configured to set an upper threshold and a lower threshold with respect to the percentage, and
the processing circuitry is configured to determine the percentage by calculating a weighted average of a value of the mean Euclidean distance at a point corresponding to the upper threshold on the MED curve and a value of the mean Euclidean distance at a point corresponding to the lower threshold on the MED curve.

5. The nuclear medicine diagnosis apparatus according to claim 3, wherein
the processing circuitry is configured to set an upper threshold and a lower threshold with respect to the percentage, and
the processing circuitry is configured to determine the percentage by identifying, on the MED curve, a point that maximizes a distance to a straight line passing a point corresponding to the upper threshold on the MED curve and a point corresponding to the lower threshold on the MED curve.

6. The nuclear medicine diagnosis apparatus according to claim 3, wherein the processing circuitry is configured to determine the percentage by calculating a second derivative with respect to the MED curve.

7. The nuclear medicine diagnosis apparatus according to claim 3, wherein
the processing circuitry is configured to set an upper threshold and a lower threshold with respect to the percentage;
the processing circuitry is configured to determine a first percentage by identifying, on the MED curve, a point that maximizes a distance to a straight line passing a point corresponding to the upper threshold on the MED curve and a point corresponding to the lower threshold on the MED curve;
the processing circuitry is configured to determine a second percentage by calculating a second derivative with respect to the MED curve; and
the processing circuitry is configured to determine the percentage by calculating a weighted average of the first percentage and the second percentage.

8. The nuclear medicine diagnosis apparatus according to claim 1, wherein the processing circuitry is configured to generate mini-frame images by reconstructing the list mode data with respect to each of the mini frames and to generate the feature vectors from the mini-frame images.

9. The nuclear medicine diagnosis apparatus according to claim 8, wherein the feature vectors are latent vectors obtained by encoding the mini-frame images.

10. The nuclear medicine diagnosis apparatus according to claim 1, wherein the processing circuitry is configured to generate the reference phase vector by averaging the feature vectors corresponding to a quiescent phase.

11. A method comprising:
generating a feature vector for each of mini frames from list mode data acquired by performing a nuclear medicine scan and calculating a Euclidean distance between each of feature vectors corresponding to frames and a reference phase vector;
calculating, on a basis of the Euclidean distances, a mean Euclidean distance for a plurality of conditions obtained by varying a percentage of counts to be used in a reconstructing process and determining the percentage on a basis of a relationship between the percentage and the mean Euclidean distance; and
generating a gated image by carrying out an image reconstruction while using the determined percentage as a gating condition.
